(19) United States
(12) Patent Application Publication
(10) Pub. No.: US 2022/0346988 A1
(43) Pub. Date: Nov. 3, 2022

(54) STENT

(71) Applicant: The University of Greenwich, Greenwich, London (GB)

(72) Inventors: Michael Okereke, London (GB); Dennis Douroumis, London (GB); Roxanne Khalaj, London (GB)

(21) Appl. No.: 17/620,243

(22) PCT Filed: Jun. 22, 2020
(86) PCT No.: PCT/GB2020/051507

§ 371 Date: Dec. 17, 2021

(30) Foreign Application Priority Data
Jun. 20, 2019 (GB) 1908855.8

(51) Int. Cl. A61F 2/915

(52) U.S. Cl. CPC A61F 2/915; A61F 2002/825

(57) ABSTRACT

The disclosure provides a tubular stent comprising at least three primary elongate columns disposed around a circumference of the stent. The primary elongate columns substantially parallel to a longitudinal axis of the stent. The stent further comprises at least two non-linear struts disposed between each pair of circumferentially adjacent primary columns, wherein each strut extends between the circumferentially adjacent primary columns. The stent is configured to adopt a first, unexpanded configuration and a second, expanded configuration in which the stent has a greater diameter than in the first, unexpanded configuration.

The invention relates to stents, especially cardiovascular stents which serve the purpose of restoring the free flow of blood within an occluded blood vessel (i.e. revascularization).

Stents are meshes used to restore structural functions of mainly cardiovascular structures. Stenting requires the insertion of the stent and balloon assembly into the occluded blood vessel during a coronary angioplasty procedure by a surgeon. The stents are usually made from metallic systems however, the stiffness mismatch between the arteries and the metals has been known to create localised damage in the blood vessel leading to restenosis (further scarring and narrowing of the blood vessel). Surgeons have become increasingly concerned about the restenosis associated with metallic stents, and there is sustained research involving stent geometric designs, material choice and new stent manufacture modalities. Recent developments of stents have explored the use of polymers in design of such stents.

Prior to use, a stent is disposed over a balloon catheter. It's then moved into an area of the blockage and the balloon is inflated, causing the stent to expand, remain in place and form a scaffold, thereby holding the artery open. Accordingly, to ensure it can be used in this manner, a stent must meet certain requirements. In particular, it must be expandable using a balloon expansion system and small enough to be inserted into the lumen without need to cut or distort the blood vessel. A stent should also experience slight elastic radial recoil (usually about 10%) following removal of the balloon pressure. A stent also must not experience undue foreshortening (i.e. reduced longitudinal recoil). Finally, the stent must remain intact during the expansion and contraction of the balloon without experiencing localised damage.

Designers have developed stents with connector rings that allow for radial concentric expansion thereof. Commercial stents have these radial connectors with varying levels of complexities. The configuration of the connector stents can determine the applicable manufacturing method as only a few manufacturing processes (usually laser cutting) can be used to manufacture stents when the geometric design is significantly complex. Such methods tend to be better adapted to metallic stents. It would be advantageous to be able to provide stents with less stiffness mismatch between the target application vascular structure and the stent material.

In recent years, research into polymer-based stents have started but are limited due to the available manufacturing methods. Accordingly, polymeric stents tend to consist of simple structures.

The present invention arises from the inventors' work in attempting to overcome the problems associated with the prior art.

In accordance with a first aspect of the invention, there is provided a tubular stent comprising:
at least three primary elongate columns disposed around a circumference of the stent, and substantially parallel to a longitudinal axis thereof; and
at least two non-linear struts disposed between each pair of circumferentially adjacent primary columns, wherein each strut extends between the circumferentially adjacent primary columns,
   wherein the stent is configured to adopt a first, unexpanded configuration and a second, expanded configuration in which the stent has a greater diameter than in the first, unexpanded configuration.

Advantageously, the inventors have found that the stent of the first aspect provides a good balance between flexibility and strength. In particular, the stent of the first aspect was found to minimise radial recoil percentage, minimise longitudinal retraction percentage and minimise foreshortening. Furthermore, the stent of the first aspect was found to have a large maximum stent stress and therefore can support increased loads and a large maximum diametral strain, and consequently can experience significant deformation without damage during the stent deployment phase.

The stent may be configured for use in a scaffolding application. The stent may be a coronary artery stent, a vascular artery stent, a biliary stent, an esophageal stent, a stent configured for use with a heart valve implantation, a pulmonary artery stent or an endovascular stent. A vascular artery stent may be a peripheral artery stent, for instance, the stent may be a carotid artery stent, an iliac artery stent, a femoral artery stent or a popliteal artery stent. The stent may be configured for use in any blood vessel of the body. Preferably, the stent is a coronary artery stent.

In the first, unexpanded configuration, the stent may define a longitudinal length of between 1 and 500 mm, more preferably between 2 and 300 mm, between 4 and 400 mm or between 6 and 200 mm. In a first embodiment, the stent may define a longitudinal length of between 5 and 100 mm or between 6 and 50 mm, and most preferably between 7 and 40 mm, between 8 and 30 mm, between 9 and 20 mm or between 10 and 15 mm. In the first embodiment, the stent may be a coronary artery stent. In a second embodiment, the stent may define a longitudinal length between 20 and 300 mm or between 50 and 250 mm, more preferably, between 100 and 225 mm or between 150 and 200 mm. In the second embodiment, the stent may be a peripheral artery stent.

The stent may be configured to convert from the first, unexpanded configuration to the second, expanded configuration. The stent may be configured to be placed over a balloon catheter. The stent may be configured to convert from the first, unexpanded configuration to the second, expanded configuration upon inflation of the balloon catheter. Alternatively, the stent may be a self-expanding or self-apposing stent. Accordingly, the stent may comprise a sheath configured to hold the stent in the first, unexpanded configuration. The stent may be configured to convert from the first, unexpanded configuration to the second, expanded configuration upon removal of the sheath.

Preferably, the tubular stent comprises between three and ten primary columns or between three and eight primary columns, more preferably between three and six primary columns or between three and five primary columns, and most preferably between three and four primary columns.

Advantageously, the number of columns may be selected depending upon the application. In some embodiments, stents comprising more columns may be advantageous. For instance, if an atherosclerotic plaque blocking an artery is large, then it may be useful to use a stent design with more coverage areas, i.e. more columns, as those will more effectively to push the plaque out of the way and hold it back, thereby increasing patency. Conversely, in other applications, it may be preferred to provide a stent comprising three or four primary columns. Advantageously, a stent with three or four columns can be made in a range of sizes. Additionally, in use, the contact area between the stent and the patient will be minimised. This has been proven to reduce the outcome of adverse effects associated with stenting, primarily restenosis and thrombosis.

In the first, unexpanded configuration, the stent may have an internal diameter of between 0.5 and 50 mm, more preferably between 1 and 40 mm or between 1.5 and 35 mm. In a first embodiment, in the first, unexpanded configuration, the stent may have an internal diameter of between 0.5 and 10 mm, more preferably between 1 and 7.5 mm, and most preferably between 1.5 and 5 mm. In the first embodiment, the stent may be a coronary artery stent. In a second embodiment, in the first, unexpanded configuration, the stent may have an internal diameter of between 2.5 and 30 mm, more preferably between 5 and 25 mm or between 7.5 and 20 mm, and most preferably between 10 and 15 mm. In the second embodiment, the stent may be a periphery artery stent or a biliary stent.

It may be appreciated that the internal diameter of the stent in the second, expanded configuration depends upon the internal diameter of the stent in the first, unexpanded configuration, the material used to form the stent and the structure of the stent.

Preferably, when the stent converts from the first, unexpanded configuration to the second, expanded configuration, the stent is configured to expand to a diametral strain of at least 50% or at least 75%, more preferably a diametral strain of at least 100%, at least 125%, at least 150% or at least 175%, and most preferably a diametral strain of at least 200%. Preferably, when the stent converts from the first, unexpanded configuration to the second, expanded configuration, the stent is configured to expand to a diametral strain of between 50% and 350% or between 75% and 325%, more preferably a diametral strain of between 100% and 300%, between 125% and 275%, between 150% and 250% or between 175% and 225%, and most preferably a diametral strain of between 200% and 210%. Alternatively, when the stent converts from the first, unexpanded configuration to the second, expanded configuration, the stent is configured to expand to a diametral strain of between 50% and 350% or between 75% and 300%, more preferably a diametral strain of between 100% and 250%, between 110% and 200%, between 120% and 175% or between 125% and 150%, and most preferably a diametral strain of between 130% and 140%.

It may be appreciated that the diametral strain is a ratio of the internal diameter of the stent in the second, expanded configuration compared to the internal diameter of the stent in the first, unexpanded configuration. Accordingly, a diametral strain of 200% would correlate to the internal diameter of the stent in the second, expanded configuration being three times greater that the internal diameter of the stent in the first, unexpanded configuration.

The stent may be configured to adopt a third, recoiled configuration in which the stent has a greater diameter than in the first, unexpanded configuration and a smaller diameter than in the second, expanded configuration. The stent may be configured to convert from the second, expanded configuration to the third, recoiled configuration. The stent may be configured to convert to the second recoiled configuration upon deflation of the balloon catheter.

It may be appreciated that the internal diameter of the stent in the third, recoiled configuration depends upon the internal diameter of the stent in the second, unexpanded configuration, the material used to form the stent and the structure of the stent.

Preferably, when the stent converts from the second, expanded configuration to the third, recoiled configuration, the stent is configured to have a radial recoil of less than 50%, more preferably less than 40%, less than 30% or less than 20%, most preferably less than 15%, less than 12.5% or less than 10%. Preferably, when the stent converts from the second, expanded configuration to the third, recoiled configuration, the stent is configured to have a radial recoil of between 1% and 50%, more preferably between 2% and 40%, between 4% and 30% or between 6% and 20%, most preferably between 7% and 15%, between 8% and 12.5% or between 9% and 10%. The radial recoil may be calculated as defined in the examples.

The external diameter of the stent will depend upon the internal diameter and the thickness of the columns and struts. The thickness may be understood to be the width of the columns and struts in a radial direction. The primary columns and struts may have a thickness between 10 and 1,000 μm, more preferably between 25 and 750 μm, and most preferably between 50 and 500 μm. In some embodiments, the primary columns and struts may have a thickness between 100 and 800 μm, between 300 and 700 μm or between 400 and 600 μm.

The stent may comprise a proximal end and a distal end. It may be appreciated that the longitudinal axis extends between the proximal and distal ends of the stent.

The non-linear struts may be disposed circumferentially between pairs of circumferentially adjacent primary columns.

Preferably, the non-linear struts each comprise a first portion and a second portion, wherein in the first, unexpanded configuration, the first and second portions may be substantially linear or geodesic. The term geodesic may be understood to mean that the first and second portions of the strut may curve with the circumference of the stent and represent the shortest path between two points on the circumference. In the first, unexpanded configuration, the first and second portions may define a first angle therebetween of between 0.5° and 189.5°, between 1° and 179° or between 5° and 175°. In the first, unexpanded configuration, the first angle may be between 10° and 150°, between 15° and 130° or between 20° and 110°, more preferably between 22.5° and 100°, between 25° and 90°, between 27.5° and 80° or between 30° and 70°, and most preferably between 32.5° and 67.5° or between 35° and 65°. In some embodiments, in the first, unexpanded configuration, the first angle may be between 20° and 150°, between 30° and 130° or between 40° and 110°, more preferably between 45° and 100°, between 50° and 90°, between 52.5° and 80° or between 55° and 70°, and most preferably between 57-5° and 67.5° or between 60° and 65°.

Each pair of circumferentially adjacent primary columns may comprise a first primary column and a second primary column, wherein the first portion of each strut disposed between the pair of circumferentially adjacent primary columns may be disposed substantially adjacent to the first primary column and the second portion of each strut disposed between the pair of circumferentially adjacent primary columns may be disposed substantially adjacent to the second primary column.

The strut may optionally comprise a bent portion. The bent portion may be disposed between the first and second portions.

In the first, unexpanded configuration, the first portion of each strut and a substantially adjacent primary column may define a second angle therebetween of between 0.25° and 89.75°, between 0.5° and 89.5° or between 1° and 89°. In the first, unexpanded configuration, the second angle may be between 5° and 85°, between 7-5° and 70°, between 10° and 60°, more preferably between 12° and 50°, between 13° and 45°, between 14° and 40° or between 15° and 37.5°, and most preferably between 16° and 35° or between 17.5° and 32.5°. In some embodiments, in the first, unexpanded configuration, the second angle may be between 7-5° and 70°, between 10° and 60°, more preferably between 15° and 50°, between 20° and 45°, between 22.5° and 40° or between 25° and 37.5°, and most preferably between 27.5° and 35° or between 300 and 32.5°. The first portion of the strut may extend between the substantially adjacent primary column and the bent portion or the second portion.

In the first, unexpanded configuration, the second portion of the strut and a substantially adjacent primary column may define a third angle of between 0.25° and 89.75°, between 0.5° and 89.5° or between 1° and 89°. In the first, unexpanded configuration, the third angle may be between 5° and 85°, between 7-5° and 70°, between 10° and 60°, more preferably between 12° and 50°, between 13° and 45°, between 14° and 40° or between 15° and 37.5°, and most preferably between 16° and 35° or between 17.5° and 32.5°. In some embodiments, in the first, unexpanded configuration, the third angle may be between 7-5° and 70°, between 10° and 60°, more preferably between 15° and 50°, between 20° and 45°, between 22.5° and 40° or between 25° and 37.5°, and most preferably between 27.5° and 35° or between 300 and 32.5°. The second portion of the strut may extend between the substantially adjacent primary column and the bent portion or the first portion.

The strut may be symmetrical. Accordingly, the second angle may be equal to the third angle.

The struts may comprise first and second opposing radial sides. A radial side may be understood to be a side which is substantially perpendicular to the longitudinal axis. The first radial side may be disposed closer to the proximal end of the stent than the second radial side. The first and second radial sides may be substantially parallel.

The struts may have a width between 10 and 1,000 μm, more preferably between 15 and 750 μm or between 20 and 500 μm. In some embodiments, the struts may have a width between 100 and 800 μm, between 200 and 600 μm or between 300 and 500 μm. The width may be understood to be the shortest distance between the first and second radial sides.

The first and second portions may directly interconnect. Accordingly, the strut may not comprise a bent portion. The strut may be angular. In the first, unexpanded configuration, the strut may define a v-shape. Accordingly, in the first, unexpanded configuration, a first radial side of the strut may define a v-shape. Additionally, in the first, unexpanded configuration, a second radial side of the strut, opposing the first radial side of the strut, may define a v-shape. In the first, unexpanded configuration, the first radial side of the first portion may be substantially linear or geodesic and the first radial side of the second portion may also be substantially linear or geodesic. In the first, unexpanded configuration, a second radial side of the first portion, opposing the first radial side of the first portion, may be substantially linear or geodesic, and the second radial side of the strut may be substantially linear or geodesic. The v-shape may point towards the distal end of the stent. The first radial side of the first portion may be substantially parallel to the second radial side of the first portion. The first radial side of the second portion may be substantially parallel to the second radial side of the second portion.

Alternatively, the strut may comprise a bent portion, and the bent portion may be curved. Accordingly, a first radial side of the bent portion may define a portion of the circumference of a circle. A second radial side of the bent portion, opposing the first radial side of the bent portion, may define a portion of the circumference of a circle. Preferably, two opposing radial sides of the bent portion define a portion of the circumference of a circle. The bent portion may define between 5 and 50% of the circumference of a circle, more preferably between 20 and 40% of the circumference of a circle, and most preferably between 30 and 35% of the circumference of a circle.

The first portion may comprise a first radial side which defines a curved section substantially adjacent to the first primary column in the adjacent pair of primary columns, and the first portion may comprise a second radial side, opposing the first radial side, which, in the first, unexpanded configuration, is substantially linear or geodesic. Alternatively, the first portion may comprise a first radial side which, in the first, unexpanded configuration, is substantially linear or geodesic, and the first portion may comprise a second radial side, opposing the first radial side, which, in the first, unexpanded configuration, defines a curved section substantially adjacent to the first primary column in the adjacent pair of primary columns. In a further alternative embodiment, the strut may comprise a second bent portion, extending between the first primary column in the adjacent pair of primary columns and the first portion. The second bent portion may be curved. Accordingly, the second bent portion may comprise first and second radial sides which are curved. The curved section of the first or second radial side of the first portion, or the second bent portion, may define an arc which is between 1 and 25% of the circumference of a circle, more preferably between 5 and 20% of the circumference of a circle, and most preferably between 10 and 15%.

The second portion may comprise a first radial side which defines a curved section substantially adjacent to the second primary column in the adjacent pair of primary columns, and the second portion may comprise a second radial side, opposing the first radial side, which, in the first, unexpanded configuration, is substantially linear or geodesic. Alternatively, the second portion may comprise a first radial side which, in the first, unexpanded configuration, is substantially linear or geodesic, and the second portion may comprise a second radial side, opposing the first radial side, which, in the first, unexpanded configuration, defines a curved section substantially adjacent to the second primary column in the adjacent pair of primary columns. In a further alternative embodiment, the strut may comprise a third bent portion, extending between the second primary column in the adjacent pair of primary columns and the second portion. The third bent portion may be curved. Accordingly, the third bent portion may comprise first and second radial sides which are curved. The curved section of the first or second radial side of the second portion, or the third bent portion, may define an arc which is between 1 and 25% of the circumference of a circle, more preferably between 5 and 20% of the circumference of a circle, and most preferably between 10 and 15%. Preferably, the second portion comprises a second radial side, opposing the first radial side, which, in the first, unexpanded configuration, is substantially linear or geodesic.

Preferably, the primary columns comprise a proximal end and a distal end. Preferably, the proximal ends of the primary columns define the proximal end of the stent. Preferably, the distal ends of the primary columns are spaced apart from the distal end of the stent.

In one embodiment, the proximal ends of the primary columns may define a point. Accordingly, first radial sides of the first and second portions of circumferentially adjacent struts, disposed substantially adjacent to the proximal end of the stent, may extend linearly until they form a point at the proximal end of the primary column disposed therebetween. In a further embodiment, the proximal ends of the primary columns may be linear. The linear proximal ends may each comprise a first radial side which extends across the column, preferably the first radial side extends across the column in a direction which is substantially perpendicular to the longitudinal axis. In a still further embodiment, the proximal ends of the primary columns may be curved. Accordingly, a first radial side of the proximal ends of the primary columns may define a curve or a portion of the circumference of a circle. The first radial side of the primary columns, and optionally portions of the first radial side of the first and second portions of circumferentially adjacent struts disposed either side of the proximal end of the column, may define between 5 and 50% of the circumference of a circle, more preferably between 20 and 45% of the circumference of a circle, and most preferably between 30 and 40% of the circumference of a circle.

In one embodiment, the distal ends of the primary columns may define a point. Accordingly, second radial sides of the first and second portions of circumferentially adjacent struts, disposed substantially adjacent to the distal end of the stent, may extend linearly until they form a point at a distal end of a primary column disposed therebetween. In a further embodiment, the distal ends of the primary columns may be linear. The linear distal ends may each comprise a second radial side which extends across the column, preferably the second radial side extends across the column in a direction which is substantially perpendicular to the longitudinal axis. In a still further embodiment, the distal ends of the primary columns may be curved. Accordingly, a second radial side of the distal ends of the primary columns may define a curve or a portion of the circumference of a circle. The second radial side of the primary columns may define between 5 and 50% of the circumference of a circle, more preferably between 20 and 45% of the circumference of a circle, and most preferably between 30 and 40% of the circumference of a circle.

The primary columns may have a width between 10 and 1,000 μm, more preferably between 20 and 750 μm or between 30 and 500 μm. In some embodiments, the struts may have a width between 100 and 800 μm, between 200 and 600 μm or between 300 and 500 μm. The width may be understood to be the shortest distance between first and second radial sides of the columns. Preferably, the first and second radial sides of the columns are substantially parallel. Preferably, the first and second radial sides of the columns are substantially linear.

Preferably, the tubular stent comprises at least 3 non-linear struts disposed between each pair of circumferentially adjacent primary columns, more preferably at least 4 or at least 5 non-linear struts disposed between each pair of circumferentially adjacent primary columns. In some embodiments, the tubular stent comprises four or five non-linear struts disposed between each pair of circumferentially adjacent primary columns. It may be appreciated that the number of struts can be increased as the length of the stent increases.

Preferably, a first non-linear strut is disposed between each pair of circumferentially adjacent primary columns, and is substantially adjacent to the proximal ends thereof. Preferably, in embodiments where the struts comprise a bent portion, the bent portion of the first non-linear strut is spaced apart from the proximal end of the stent.

Preferably, a second non-linear strut is disposed between each pair of circumferentially adjacent primary columns, and is substantially adjacent to the distal ends thereof. Preferably, the second non-linear strut defines the distal end of the stent. In embodiments where the struts comprise a bent portion, the bent portion of the second non-linear strut defines the distal end of the stent.

Preferably, one or more further non-linear struts is disposed between each pair of circumferentially adjacent primary columns, and is disposed between the proximal and distal ends thereof. Preferably, the one or more further non-linear struts is spaced substantially equidistantly between longitudinally adjacent struts. Substantially equidistantly may be understood to mean no more than 25% closer to one longitudinally adjacent strut than another, preferably no more than 10% closer to one longitudinally adjacent strut than another, and most preferably no more than 5% closer to one longitudinally adjacent strut than another.

Preferably, each non-linear strut is substantially parallel to any longitudinally adjacent struts. Accordingly, the first portion of a strut may be substantially parallel to the first portion of any longitudinally adjacent struts. Similarly, the second portion of a strut may be substantially parallel to the second portion of any longitudinally adjacent struts.

In some embodiments, the stent may comprise at least three secondary elongate columns disposed around the circumference of the stent, and substantially parallel to the longitudinal axis thereof, wherein the secondary columns intersect the struts.

Preferably, the secondary columns intersect the stents between the first and second portions. Optionally, the secondary columns intersect the bent portion of the struts. In embodiments where the secondary columns are present, preferably the stent comprises as many secondary columns as primary columns. Preferably, the secondary columns comprise a proximal end and a distal end. Preferably, the distal ends of the secondary columns define the distal end of the stent. Preferably, the proximal ends of the secondary columns are spaced apart from the proximal end of the stent.

The width, thickness and/or length of the secondary columns may be substantially the same as the width, thickness and/or length of the primary columns.

In one embodiment, the distal ends of the secondary columns may define a point. Accordingly, second radial sides of the first and second portions of circumferentially adjacent struts, disposed substantially adjacent to the distal end of the stent, may extend linearly until they form a point at the distal end of the secondary column disposed therebetween. In a further embodiment, the distal ends of the secondary columns may be linear. The linear distal ends may each comprise a second radial side which extends across the secondary column, preferably the second radial side extends across the secondary column in a direction which is substantially perpendicular to the longitudinal axis. In a still further embodiment, the distal ends of the secondary columns may be curved. Accordingly, a second radial side of the distal ends of the secondary columns may define a curve or a portion of the circumference of a circle. The second radial side of the secondary columns, and optionally portions of the second radial sides of the first and second portions of circumferentially adjacent struts disposed either side of the distal end of the secondary column, may define between 5 and 50% of the circumference of a circle, more preferably between 20 and 45% of the circumference of a circle, and most preferably between 30 and 40% of the circumference of a circle.

In one embodiment, the proximal ends of the secondary columns may define a point. Accordingly, first radial sides of the first and second portions of circumferentially adjacent struts, disposed substantially adjacent to the proximal end of the stent, may extend linearly until they form a point at a proximal end of the secondary column disposed therebetween. In a further embodiment, the proximal ends of the secondary columns may be linear. The linear proximal ends may each comprise a first radial side which extends across the column, preferably the first radial side extends across the column in a direction which is substantially perpendicular to the longitudinal axis. In a still further embodiment, the proximal ends of the secondary columns may be curved. Accordingly, a first radial side of the proximal ends of the secondary columns may define a curve or a portion of the circumference of a circle. The first radial side of the secondary columns may define between 5 and 50% of the circumference of a circle, more preferably between 20 and 45% of the circumference of a circle, and most preferably between 30 and 40% of the circumference of a circle.

The stent may be a drug-eluting stent. Drug-eluting stents are suitable for treating diseases or trauma that require therapeutic assistance while the vessel is being healed (i.e. during the healing window). Accordingly, the stent may comprise a therapeutic agent. The stent may comprise a drug-eluting coating comprising the therapeutic agent.

The stent may comprise a coating disposed around the outer circumference of the stent. Accordingly, the coating may define a tube around the stent. The coating may be a polymer or fabric coating.

In some embodiments, the stent does not comprise secondary columns. Accordingly, the stent may consist of the primary columns and the struts, and optionally the drug-eluting coating and/or polymer or fabric coating.

The stent may comprise a polymeric material and/or a metal. In particular, the columns and/or struts may comprise a polymeric material and/or a metal. The polymeric material may comprise a biocompatible polymer, a biodegradable polymer and/or a bioresorbable polymer. Examples of suitable polymeric materials include polylactic acid (PLA), polydioxanone (PDO), polycaprolactone (PCL), poly(lactide-co-glycolide acid) (PLGA), polyanhydrides, poly(methyl methacrylate) (PMMA), chitosan, polyurethane (PUR), hydroxypropylmethylcelulose (HPMC), gelatine or a combination thereof. It may be appreciated that polylactic acid can include both poly(l-lactic acid) (PLLA) and poly(d-lactic acid) (PDLA). The metal may comprise or be a biocompatible metal, such as stainless steel, iron, zinc, tantalum, platinum, magnesium, cobalt-chromium and/or an alloy thereof, and/or nitinol alloy. The metal may comprise or be a biocompatible and biodegradable metal, such as iron, zinc, magnesium and/or an alloy thereof.

In some embodiments, the stent may comprise a polymer matrix with metallic tracers disposed therein. Advantageously, the metallic tracers allow for x-ray guided tracing of the position of the stent during percutaneous introduction into an occluded artery.

The stent may be manufactured using three-dimensional (3D) printing technologies, injection moulding or compression moulding. Suitable 3D printing technologies include: Fused Deposition Modelling (FDM)/Fused Filament Fabrication (FFF) or other extrusion based technique, Selective Laser Sintering (SLS) or other Powder Bed Fusion (PBF) technologies, Stereolithography (SLA) or other vat polymerisation or liquid polymer system technology, Material Jetting/Polyjetting or Drop on Demand technologies. FDM/FFF may comprise printing upon a flat build plate or upon a rotating mandrel.

Alternatively, the stent may be manufactured by cutting the stent design into a wire, tube, sheet or block of material. The material could be cut using a laser or an abrasive water jet.

In a second aspect, there is provided a method of treating a vascular disease in a subject in need of such treatment, the method comprising fitting the stent according to the first aspect into a vessel of the subject.

The vascular disease may be stenosis, restenosis, thrombosis, hypertension, hemophilia, angioedema, hyperlipidemia, vasculitis, peripheral vascular disease, an aneurysm or an intracranial aneurysm.

The vessel may be a blood vessel, the esophagus or form part or the biliary tract. The blood vessel may be an artery, an arteriole, a vein or a venule. The artery may be a coronary artery or a peripheral artery. The peripheral artery may be a carotid artery, an iliac artery, a femoral artery or a popliteal artery. Alternatively, the vessel may be a bile duct. Preferably, the vessel is a coronary artery.

The subject may be a human or an animal. Preferably, the subject is a mammal, and more preferably a human.

All of the features described herein (including any accompanying claims, figures and abstract), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying FIGS., in which:—

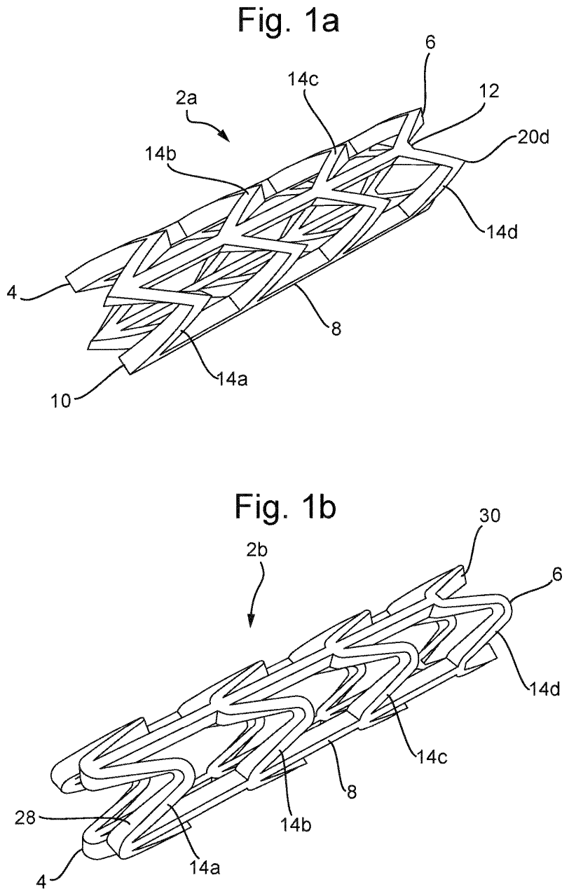

FIG. 1 is a three-dimensional representation of stents defining (a) angular and (b) curved chevron-shaped cell structures;

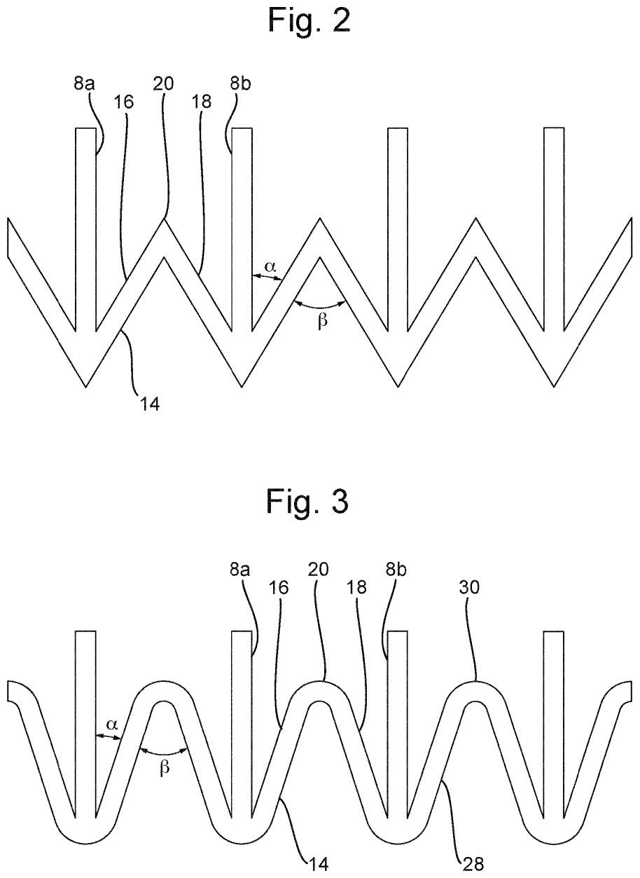

FIG. 2 shows a line of repeating units of the angular chevron-shaped cell stent of FIG. 1a;

FIG. 3 shows a line of repeating units of the curved chevron-shaped cell stent of FIG. 1b;

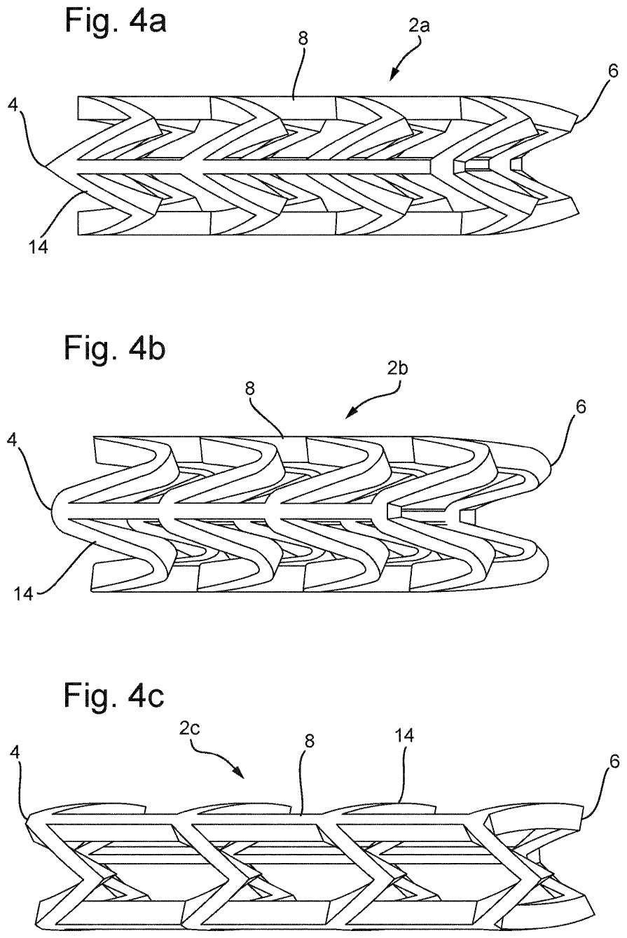

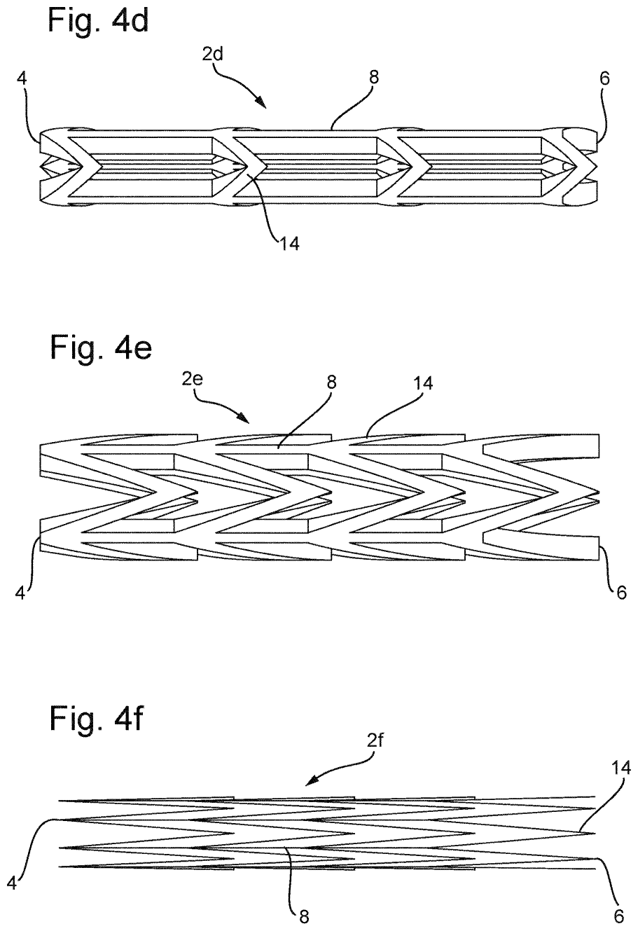

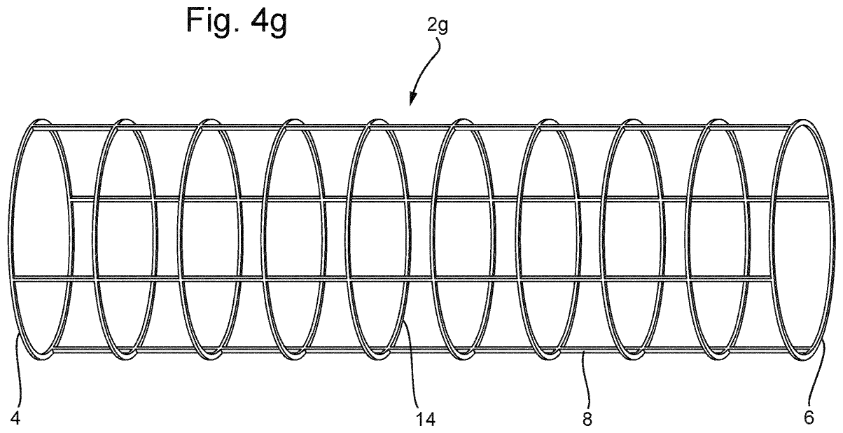

FIG. 4a shows an isometric view of the stent of FIG. 1a; FIG. 4b shows an isometric view of the stent of FIG. 1b; and FIGS. 4c-g show isometric views of further stents in accordance with the present invention;

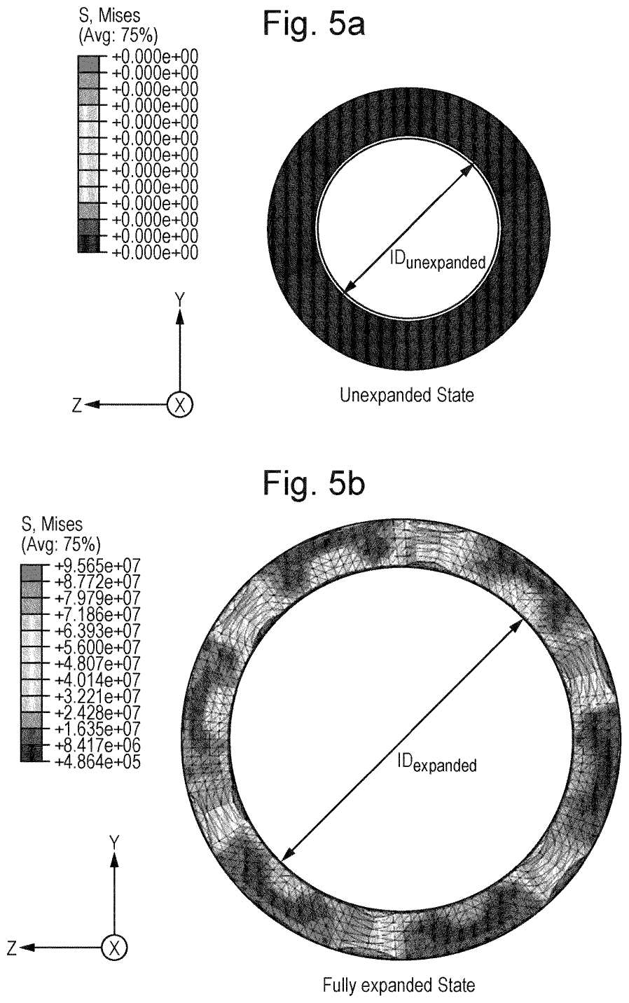

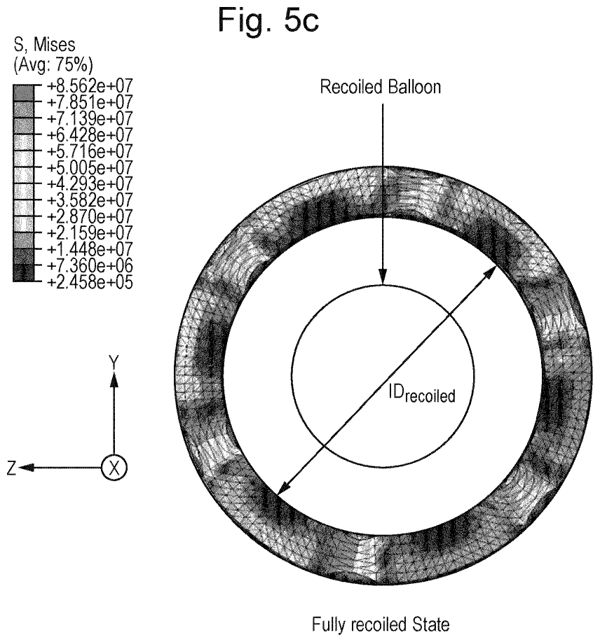

FIG. 5 shows contour plots for the stent shown in FIG. 4e in the (a) unexpanded, (b) fully expanded, and (c) fully recoiled states, when viewed along the longitudinal axis. The insert legend gives a measure of the effective stress state within the stent material based on polydioxanone (PDO) polymeric material;

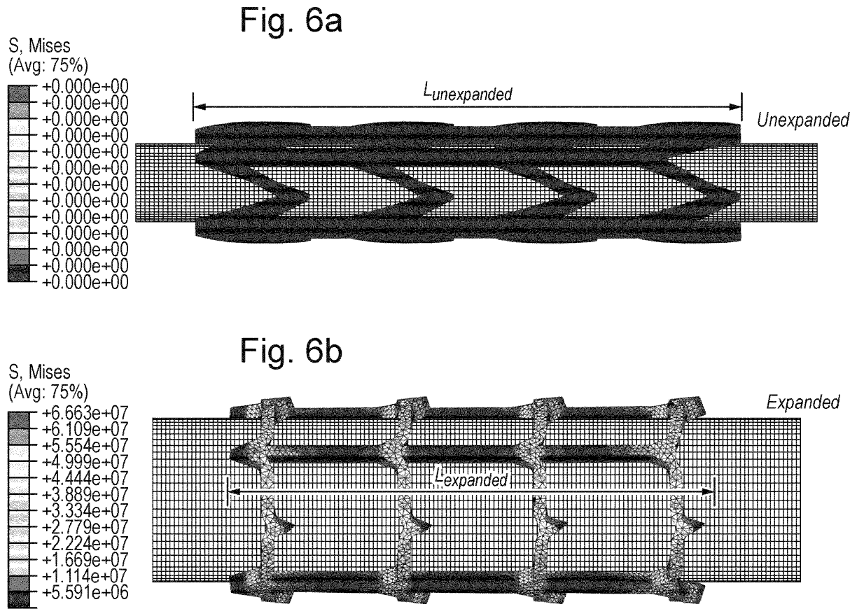

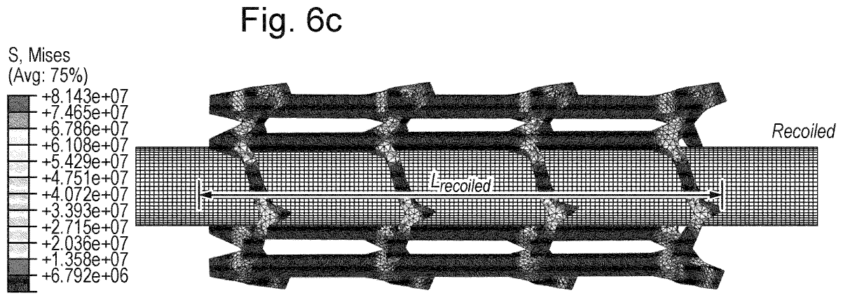

FIG. 6 shows contour plots for the stent shown in FIG. 4e in the (a) unexpanded, (b) fully expanded, and (c) fully recoiled states, when viewed perpendicular to the longitudinal axis. The insert legend gives a measure of the effective stress state within the stent material based on polydioxanone (PDO) polymeric material;

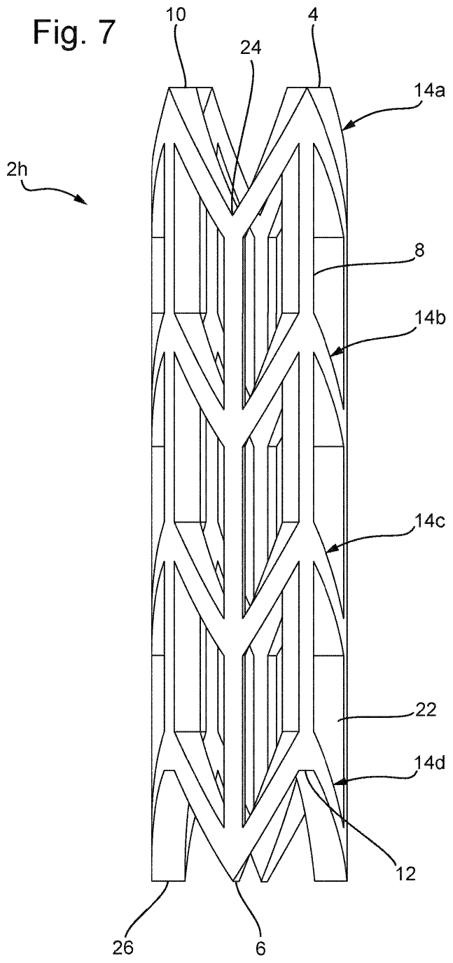

FIG. 7 is a three-dimensional representation of an alternative stent;

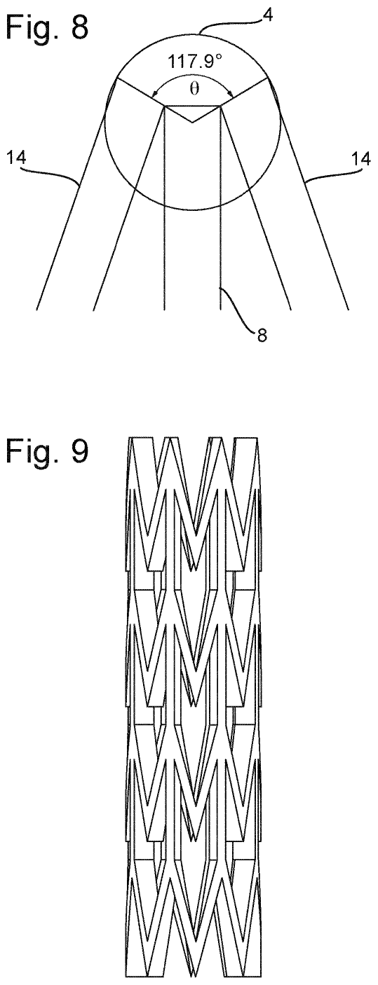

FIG. 8 shows a portion of the stent of FIGS. 1b and 4b;

FIG. 9 is a virtual designs of a stent;

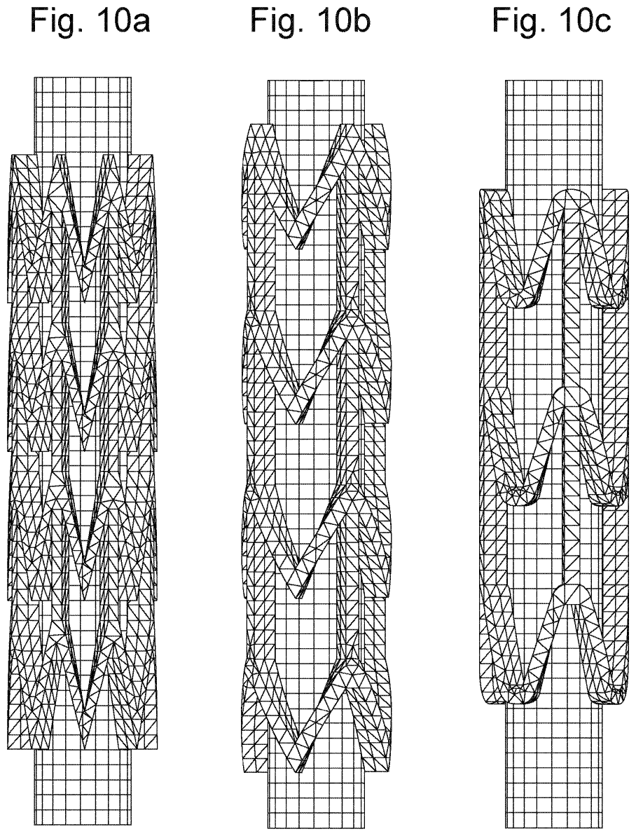

FIG. 10 shows the assembled ex-vivo setup of three stents showing balloon and the stents: (a) is the stent shown in FIG. 9, identified as Stent 1, (b) is the stent shown in FIG. 1a, identified as Stent 2, and (c) is the stent shown in FIG. 1c, identified as Stent 3;

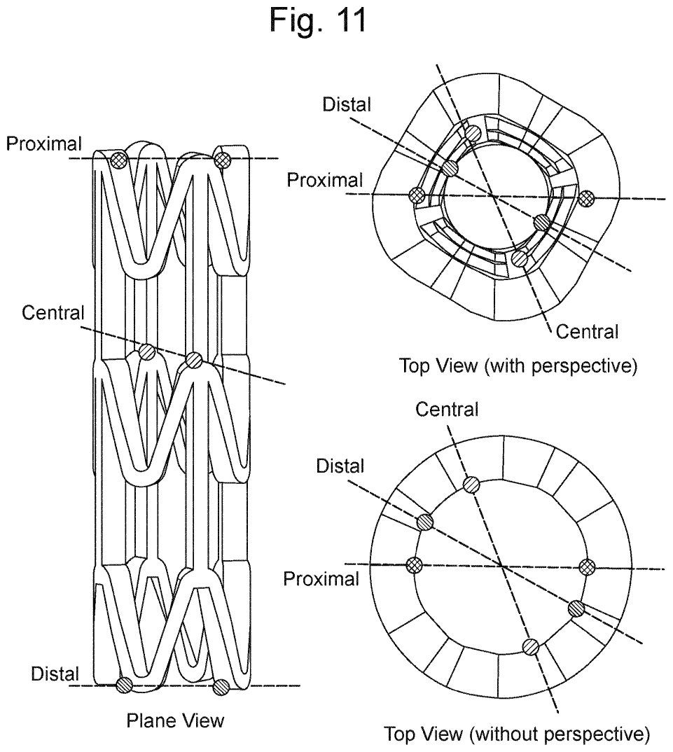

FIG. 11 is an illustration, using Stent 3, of three regions at which structural parameters were extracted for the three stents. These regions coincide with proximal, central and distal regions of the stents, along the line of insertion in the occluded artery. For each region, two reference points were identified and kinematically tracked during the deformation;

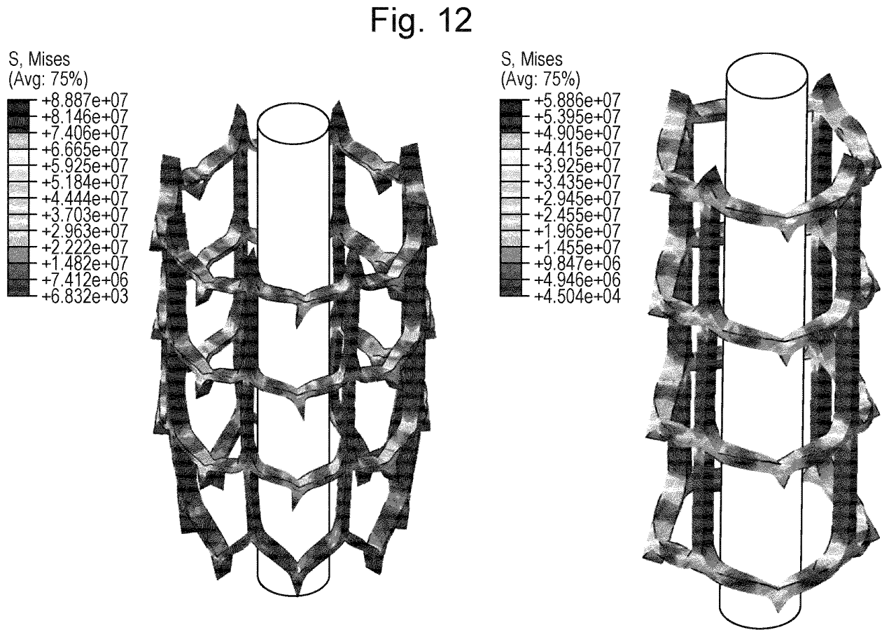

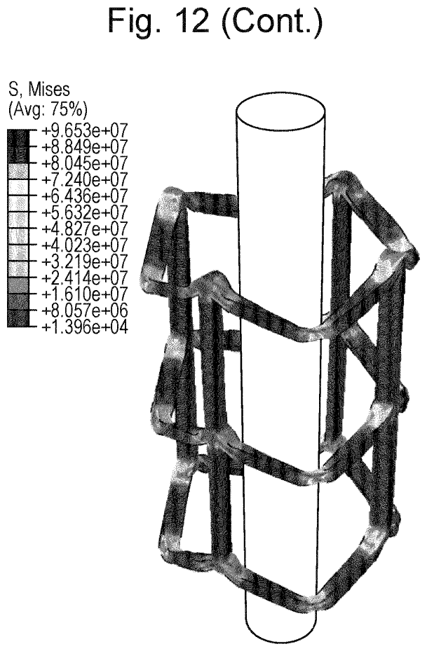

FIG. 12 shows stent mechanics contour plots for the three stents showing stress distribution in the stents for (a) Stent 1, (b) Stent 2, and (c) Stent 3;

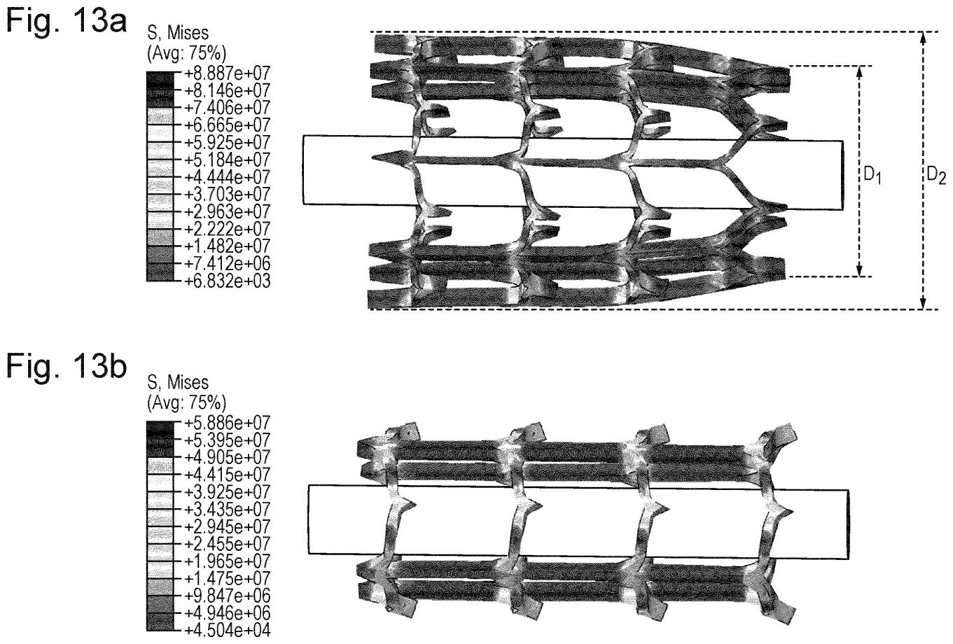

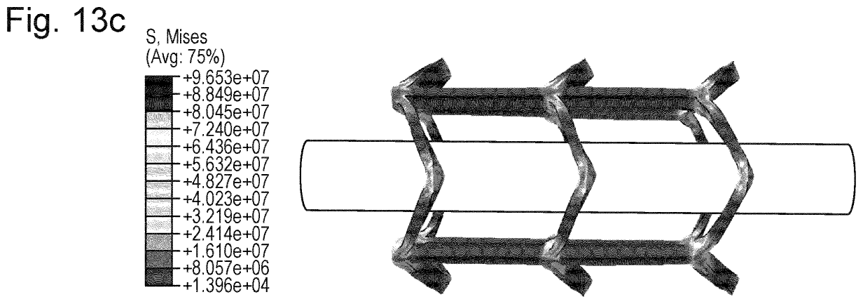

FIG. 13 shows evidence of dogboning from contour plots from the virtual testbed showing (a) significant dogboning of Stent 1 with D₂>>D₁, (b) limited dogboning for Stent 2, and (c) limited dogboning for Stent 3;

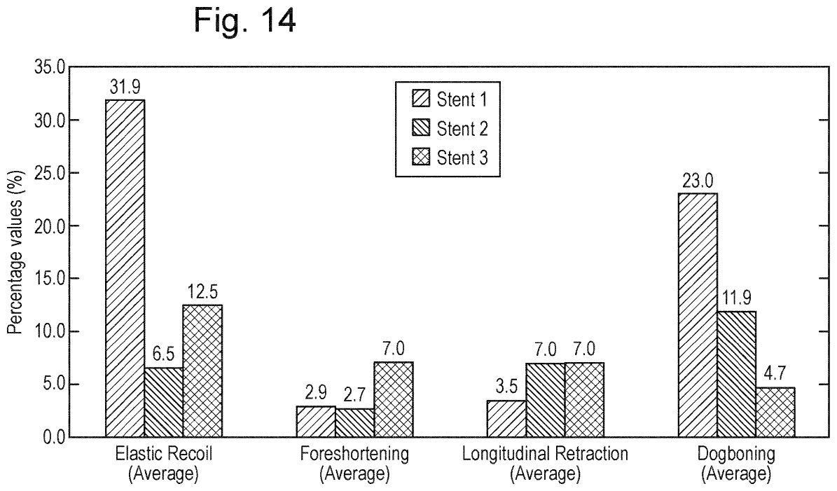

FIG. 14 is a graph comparing the structural parameters of Stents 1 to 3;

### EXAMPLE 1—DESIGN OF A NEW STENT

A tubular stent 2a is shown in an unexpanded configuration in FIGS. 1a and 4a. The stent 2a comprises a proximal end 4 and a distal end 6, and a longitudinal axis extends therebetween. The tubular stent 2 has a substantially cylindrical cross-section with an internal diameter of about 2 mm, an outer diameter of about 3 mm and a total length of about 12 mm.

The stent 2a comprises four elongate columns 8, each column 8 comprising a proximal end 10 and a distal end 12. The proximal end 10 of each column 8 is angular, defining a point. Conversely, the distal end 12 of each column 8 is linear, defining a surface substantially perpendicular to the longitudinal axis of the stent 2a. The columns 8 have a depth of approximately 0.5 mm, a width of approximately 0.3 mm and a length of about 9.75 mm. The columns are disposed parallel to the longitudinal axis of the stent 2, and are evenly spaced around the circumference of the tubular stent 2. The proximal ends 10 of the columns 8 are level with and define the proximal end 4 of the stent 2. Conversely, the distal ends 12 of the columns 8 are spaced apart from the distal end 6 of the stent 2a.

As shown in FIG. 2, a bent strut 14 extends between adjacent columns 8a, 8b. The strut comprises a bent portion 20, a first portion 16, extending between the first column 8a and the bent portion 20, and a second portion 18, extending between the second column 8b and the bent portion 20. The first and second portions 16, 18 each define an angle α with the adjacent column 8a, 8b and an angle β with each other. In the embodiment shown in FIGS. 1 and 2, a is 31.31° and β is 62.62°. The first and second portions 16, 18 have a length of about 2.3 mm and have a width of about 0.3 mm and a depth of approximately 0.5 mm.

As shown in FIG. 1, four struts 14 extend circumferentially between each pair of circumferentially adjacent columns 8. A first strut 14a is disposed adjacent to the proximal ends 10 of the columns 8. Similarly, a second strut 14b is disposed a third of length along the columns 8, closer to the proximal ends 10 thereof. A strut 14c is located two thirds of length along the columns 8, closer to the distal ends 12 thereof. Finally, a fourth strut 14d is located adjacent to the distal ends 12 of the columns 8. The four struts 14 disposed between each pair of columns 8 are all parallel to each other. The bent portions 10 of all the struts 14 point towards the distal end 6 of the stent. In particular, the bent points 20d of the fourth strut 14d define the distal end 6 of the stent 2a.

The stent 2a shown in FIGS. 1a and 4a has an angular configuration i.e., as explained above, the proximal ends 10 of the columns 8 define points and the bent portions 20 also define points.

An alternative stent 2b is shown in FIGS. 1b and 4b, and FIG. 3 shows a line of repeating units for this stent 2b. The stent 2b has a substantially cylindrical cross-section with an internal diameter of about 2 mm, an outer diameter of about 3 mm and a total length of about 10 mm. In this embodiment, the columns define a length of about 7.5 mm. Additionally, in this embodiment, the angle α is 18.74° and the angle β is 37.48°. The first and second portions 16, 18 have a length of about 1.9 mm, and both the columns 8 and the struts 14 have a width of approximately 0.3 mm and a depth of approximately 0.5 mm.

This stent 2b is similar to the stent 2a shown in FIGS. 1a and 2 but has a curved configuration. Accordingly, in this embodiment, the proximal ends 10 of the columns 8 define curved surfaces. This curved portion of the design can be defined by the are length of the curved surface using the following equation:

Arc length=(θ/360)×π×D

θ is the angle shown in FIG. 8, and D is the diameter of the circle. The stent shown in FIGS. 1b and 4b has an arc length of 0.9877 mm, which is 32.75% of the circumference of the circle. However, it will be appreciated that the are length, and the portion of a circle the proximal end 10 of the column 8 defines will vary as the angles α and β vary.

Similarly, bent portions 20, where the first and second portions 16, 18 meet, also define curved surfaces which define 32.75% of the circumference of the circle. As shown in FIGS. 1b and 4b, a proximal radial side 28 of the first and second portions 16, 18 is curved adjacent to the column 8. A distal radial side 30 of the first and second portions 16, 18 is linear adjacent to the column 8 in the pictured stent, but it will be appreciated that in some embodiments the distal radial side of the first and second portions 16, 18 could be curved.

Alternative stents with angular configurations are shown in FIGS. 4c to 4g. These stents have been modified by changing the size of the columns, the number of columns, the size of the struts, the number of struts repeating in a longitudinal direction and/or the angles of α and β. Furthermore, the stents shown in FIGS. 4c and 4d comprise three (as opposed to four) columns 8.

Briefly, a stent 2c shown in FIG. 4c comprises three columns 8 and four pairs of struts 14 are disposed between each circumferentially adjacent pair of columns 8. The stent 2c has a length of 11.97 mm, an inner diameter of 1.5 mm and the struts 14 and columns 8 have a depth of 0.5 mm and a width of 0.3 mm, a is 37.69° and β is 75.38°.

A stent 2d shown in FIG. 4d comprises three columns 8 and four pairs of struts 14 are disposed between each circumferentially adjacent pair of columns 8. The stent 2d has a length of 12.01 mm, an inner diameter of 0.7 mm and the struts 14 and columns 8 have a depth of 0.5 mm and a width of 0.3 mm, α is 440 and β is 88°.

A stent 2e shown in FIG. 4e comprises four columns 8 and four pairs of struts 14 are disposed between each circumferentially adjacent pair of columns 8. The stent 2e has a length of 11.99 mm, an inner diameter of 1.7 mm and the struts 14 and columns 8 have a depth of 0.5 mm and a width of 0.3 mm, a is 20.01° and β is 44.01°.

A stent 2f shown in FIG. 4f comprises eight columns 8 and four pairs of struts 14 are disposed between each circumferentially adjacent pair of columns 8. The stent 2f has a length of 22.31 mm, an inner diameter of 2.9 mm and the struts 14 and columns 8 have a depth of 0.05 mm and a width of 0.05 mm, a is 5° and β is 10°.

A stent 2g shown in FIG. 4g comprises four columns 8 and ten pairs of struts 14 are disposed between each circumferentially adjacent pair of columns 8. The stent 2g has a length of 14 mm, an inner diameter of 4.21 mm and the struts 14 and columns 8 have a depth of 0.09 mm and a width of 0.03 mm, a is 89.5° and β is 179°.

A further alternative stent 2h is shown in FIG. 7. This stent 2h has an angular configuration, and is similar to the stent 2a depicted in FIGS. 1a and 4a. However, the stent further comprises four secondary columns 22, each secondary column 22 comprising a proximal end 24 and a distal end 26. The proximal end 24 of each secondary column 22 is angular, defining a point. The distal end 24 of each secondary column 22 is also angular, defining a point.

The secondary columns 22 have a depth and width of approximately 0.5 mm and a length of about 9.75 mm. The secondary columns 22 are disposed parallel to the longitudinal axis of the stent 2, and are evenly spaced around the circumference of the tubular stent 2, wherein each secondary column 22 is evenly spaced between a pair of primary columns 8. Accordingly, the secondary columns 22 intersect the bent portions of the stents 14.

The proximal ends 24 of the secondary columns 22 are spaced apart from the proximal end 4 of the stent 2. Conversely, the distal ends 26 of the secondary columns 22 define the distal end 6 of the stent 2.

While the distal ends 12 of the columns 8 are shown as linear in the FIGS., it will be appreciated that the stents could be modified such that the distal end 12 is angular or curved.

### EXAMPLE 2—COMPUTATIONAL ASSESSMENT OF THE PROPOSED STENTS' STRUCTURAL RESPONSE

### Methods

Finite element analysis (FEA) computational studies were conducted on several stent designs some based on more traditional stent types in comparison with the proposed stent design. Out of different stents studied, the angular chevron-shaped cell stent and curved chevron-shaped cell stent performed consistently, in comparison with existing stents.

This study was carried out using a virtual testing framework developed by the inventors, whose computational engine is based on a finite element modelling scheme. The framework was configured to model deployment of the stent using a balloon catheter during percutaneous transluminal angioplasty (PCTA). Accordingly, the framework modelled an unexpanded stent as being disposed around a balloon catheter. The balloon was then inflated up to 0.97 mm radial displacement over 0.45 seconds, held at maximum radial expansion for 0.1 second and then deflated at the same over 0.45 seconds to its initial diameter. During the procedure, time-dependent kinematic and kinetic outputs were recorded, and post-test were collated and used to assess the structural performance of the stent. The studies were carried out using a stent comprising a polydioxanone (PDO) polymeric material. The computational studies comprised only the stent design and a balloon structure. This representative volume element of the coronary angioplasty system was chosen to comprise of only these two materials to reduce the computational cost for the study and emphasize solely the impact of the stent geometries on the stent design.

The structural performance was assessed based on accepted stent mechanical response parameters, namely: (1) radial recoil, (2) longitudinal retraction, (3) foreshortening, (4) maximum stent stress and (5) maximum strain. The stent parameters are objective measures of the structural response of the stent with respect to the desired stent expansion and deflation mechanics.

### 1. Radial Recoil

Radial recoil percentage (R_{recoil}) is a percentage of the ratio of change in internal diameter of the stent following deflation of the balloon in comparison to its fully expanded internal diameter. It may be calculated using the following formula:

$${R}_{r ecoil} = \frac{{ID}_{expanded} - {ID}_{r ecoiled}}{{ID}_{expanded}} \times 100 \%$$

Where ID_{expanded} is the internal diameter of the stent when it is in the expanded state and ID_{recoiled} is the internal diameter of the stent after the balloon has deflated.

It is desirable for the radial recoil percentage to be minimised, preferably to within approximately 10%.

### 2. Longitudinal Retraction

Longitudinal retraction (L_{retraction}) is a measure of the ratio of change in the total axial stent length when expanded, compared to the original length of the unexpanded stent. It may be calculated using the following formula:

$${L}_{r e traction} = \frac{{L}_{u nexpanded} - {L}_{expanded}}{{L}_{unexpanded}} \times 100 \%$$

L_{unexpanded} and L_{expanded} refer to the axial length of the stent in the unexpanded and expanded configurations, respectively.

It is also desirable for the longitudinal retraction to be minimised, preferably to within approximately 10%.

### 3. Foreshortening

Foreshortening (Fₛₕ) is related to the radial recoil percentage and refers to the ratio of the change in axial length of the stent between the recoiled position and the fully expanded position. It gives an indication of the lateral recoil of the stent following deflation of the balloon. It may be calculated using the following formula:

$${F}_{s h} = \frac{{L}_{r e c o i l e d} - {L}_{expanded}}{{L}_{expanded}} \times 100 \%$$

L_{recoiled} refers to the axial length of the stent after the balloon has deflated.

It is also desirable for the foreshortening to be minimised, preferably to within approximately 10%.

### 4. Maximum Stent Stress

Maximum stent stress is a measure of the largest stress that the stent can support under the effect of an inflating balloon. It represents the maximum average stress state within the balloon. It also gives an indication of the load-bearing capacity of the stent. For this study the stress recorded was von Mises stress.

This is an important measure as it illustrates the possibility of the stent to fail/damage during the balloon inflation process. A comparison of this stress with say the yield stress or ultimate tensile strength of the test material (PDO for example) will help you know if the effective stress experienced by the stent material is ‘dangerously’ close to the failure load of the test material. For such cases, the stent will be highly susceptible to failure which is not good.

It is not the same for all the stent parts because the stent is a complex ‘mesh-like structure’ so the distribution of load within them will be different hence leading to different stress states shown from the computational framework.

This structural response helps select which material, design combination and inflating pressure that will be suitable for the coronary angioplasty while ensuring the structure is still intact and not susceptible to failure.

It is desirable for the maximum stent stress to be maximised, preferably up to the yield stress of the stent material.

### 5. Maximum Diametral Strain

Maximum diametral strain (Øε) is a measure of the maximum strain experienced by the stent following the balloon deployment. It is a structural strain measured radially along the diameter of the stent. It is an indicator of the ability of the stent to support the imposed balloon pressure without structural collapse of the stent due to tensile failure in one of the struts. Higher strain without localised damage/over-stretching of the stent is most desirable. It may be calculated using the following formula:

$${∅ε}_{max} = \frac{{∅}_{expanded} - {∅}_{unexpanded}}{{∅}_{expanded}} \times 100 \%$$

Where Ø_{expanded} and Ø_{unexpanded} are the expanded and unexpanded stent internal diameters, respectively.

It is also desirable for the maximum diametral strain to be maximised.

### Results

FIGS. 5 and 6 show the contour plots from the virtual testing scheme for a stent in the three deformation states (unexpanded, expanded and recoiled). The legend gives a measure of the effective stress state within the stent material based on polydioxanone (PDO) polymeric material. The results are shown in Table 1.

TABLE 1
Calculated structural stent parameters

| | | | | Maximum | | |
|---|---|---|---|---|---|---|
| | Radial | Longitudinal | | Diametral | Maximum | |
| Stent | Recoil | Retraction | Foreshortening | Strain | Stress | |
| Angular chevron stent, | 9.94% | 8.17% | 2.60% | 128.86% | 81.43 | MPa |
| as shown in Figures | | | | | | |
| 1a and 4a | | | | | | |
| Curved chevron stent, | 10.45% | 15.48% | 5.40% | 206.08% | 71.7 | MPa |
| as shown in Figures | | | | | | |
| 1b and 4b | | | | | | |

Across all tested stent structural parameters, except maximal diametral strain, the angular chevron stent seemed to out-perform the curved chevron stent. The former also meets with the requirements for a good stent design and compares favourably with metallic stents in all parameters.

The present design compared favourably to commercially available stents. For instance, in silico studies done by Schiavone et al. (2017) show recoiling effect for commercial stents, Xience was 11% and for Elixir DESolve, which is fully polymeric was 20%.

The angular chevron cell experienced just over 100% maximum diametral strain, unlike the curved chevron cell stent which experienced over 200% strain. This indicates that the curved chevron stent has a higher expansion potential and can be used in cases were larger strains are required. Both stents have structural values in line with the structural stent parameters for commercially available stents.

### EXAMPLE 3—FURTHER COMPUTATIONAL ASSESSMENT OF THE PROPOSED STENTS' STRUCTURAL RESPONSE

### Methods

Three stents were designed in SolidWorks and imported into the virtual testbed for analysis. The stents are shown in FIG. 9 and FIGS. 1a and 1b. The stent shown in FIG. 9, hereinafter identified as “Stent 1”, has six struts and the angle at position a is 15°. The stents shown in FIGS. 1a and 1b are described above, and hereinafter are identified as “Stent 2” and “Stent 3”, respectively.

The stents were set up as shown in FIG. 10 for virtual testing. FIG. 11 shows the regions where structural parameters were extracted and FIG. 12 shows stent mechanics contour plots which were generated for the three stents.

### Dogbone Assessment

The inventors reviewed the dogbone response of the stents, as its vital that stents that should be deployed within an occluded artery must show limited dogbone effects. The contour plots for all three stents (without perspective) are shown in FIG. 13.

As shown in the contour plots, Stent 1 is subject to asymmetric dogboning effects with only the distal ends showing least expansion while the proximal and medial/central regions deformed comparably. This sort of stent mechanics is not suitable for a stent for use within the arteries as this will lead to differential/variable scaffolding effects at different regions of the arteries.

### Structural Mechanics

Structural mechanics were extracted using the reference nodes at the distal, central and proximal regions. The following structural parameters were extracted:
Elastic (or Radial) Recoil
Longitudinal retraction
Foreshortening
Dogboning
Stent mechanics assessment—which assesses if stent is suitable for in vivo use
Damage assessment index: has any of the stent's struts damaged or not.

The results are shown in Table 2.

TABLE 2
Comparison of structural parameters for three stent designs

| | | Stent Type | | |
|---|---|---|---|---|
| Structural Parameters | Units | Stent 1 | Stent 2 | Stent 3 |
| Maximum radial | mm | 2.6 | 1.3 | 1.9 |
| displacement | | | | |
| Maximum Diametral Strain | % | 274.2 | 137.8 | 199.5 |
| Elastic Recoil (Average) | % | 31.9 | 6.5 | 12.5 |
| Foreshortening (Average) | % | 2.9 | 2.7 | 7.0 |
| Longitudinal | % | 3.5 | 7.0 | 7.0 |
| Retraction (Average) | | | | |
| Dogboning (Maximum) | % | 23.0 | 11.9 | 4.7 |
| Damage Assessment | — | PASSED | PASSED | PASSED |
| Index (DAI) | | | | |
| Stent Mechanics | — | NO | YES | YES |
| Assessment (SMA) | | | | |
| Ranking | — | 3^{rd} | 1^{st} | 2^{nd} |

A comparison of the structural parameters is shown in FIG. 14.

### Findings

The numerical study has shown the following:
a) Stents 2 and 3 (Curved and Straight versions) are suitable for deployment within the occluded artery without experiencing any damage of the struts even up to 130% diametral strain.
b) Stent 1 behaved poorly in terms of structural parameters such as elastic radial recoil (32%) in comparison with the Stents 2 and 3 which showed values of 6.5% and 12.5% respectively for the Straight and Curved Chevron versions.
c) Also, the Stent 1 showed significant dogboning with maximum values of 23% whilst Stents 2 and 3 showed values of 12% and 5% respectively. Dogboning is a feature of many stents and the objective is to make sure it is minimized as such high values as 23% will lead to variable scaffolding of the artery after stent deployment which can lead to structural and arterial failures in the long term.
d) All three stents showed acceptable longitudinal retraction and foreshortening properties.
e) Stent 1 showed the largest diametral expansion experiencing as much as 274% strain whilst Stent 2 gave the least value of 138% and Stent 3 a value of 200%.
f) The stent-to-artery contact area is lower for Stents 2 and 3 than that of Stent 1 which significantly improves their use as it will allow for less arterial contacts and consequent arterial stress during the deployment.

### CONCLUSIONS

The novel design of Stents 2 and 3 makes them significantly better than existing competitor stents since they:
a. Limit dogboning effects;
b. Limit elastic recoil, an essential feature for effective scaffolding of the artery post-deployment of the stent; and
c. Limit the stent-to-artery contact area allowing for better stent mechanics, less arterial contacts and consequent reduction in arterial stress during stent deployment.

1. A tubular stent comprising:
at least three primary elongate columns disposed around a circumference of the stent, and substantially parallel to a longitudinal axis thereof, and
at least two non-linear struts disposed between each pair of circumferentially adjacent primary columns, wherein each strut extends between the circumferentially adjacent primary columns,
wherein the stent is configured to adopt a first, unexpanded configuration and a second, expanded configuration in which the stent has a greater diameter than in the first, unexpanded configuration.

2. The tubular stent according to claim 1, wherein the stent defines a longitudinal length of between 1 and 500 mm.

3. The tubular stent according to claim 1, wherein the tubular stent comprises between three and four primary columns.

4. The tubular stent according to claim 1, wherein, in the first, unexpanded configuration, the stent has an internal diameter of between 0.5 and 50 mm.

5. The tubular stent according to claim 1, wherein the stent is configured to convert from the first, unexpanded configuration to the second, expanded configuration, and when the stent converts from the first, unexpanded configuration to the second, expanded configuration, the stent is configured to expand to a diametral strain of at least 50%.

6. The tubular stent according to claim 1, wherein the stent is configured to convert from the second, expanded configuration to a third, recoiled configuration in which the stent has a greater diameter than in the first, unexpanded configuration and a smaller diameter than in the second, expanded configuration, and when the stent converts from the second, expanded configuration to the third, recoiled configuration, the stent is configured to have a radial recoil of less than 50%.

7. The tubular stent according to claim 1, wherein the primary columns and struts have a thickness between 10 and 1,000 μm.

8. The tubular stent according to claim 1, wherein the struts each comprise a first portion and a second portion, wherein in the first, unexpanded configuration, the first and second portions are substantially linear or geodesic, and the first and second portions of each strut define a first angle therebetween of between 0.5° and 189.5°.

9. The tubular stent according to claim 8, wherein the first portion of the strut and a substantially adjacent primary column define a second angle therebetween, and the second portion of the strut and a substantially adjacent primary column define a third angle therebetween, and in the first, unexpanded configuration, the second and third angles are both between 0.25° and 89.75°.

10. The tubular stent according to claim 8, wherein the first and second portions of the strut directly interconnect.

11. The tubular stent according to claim 8, wherein, in the first, unexpanded configuration, the strut defines a v-shape.

12. The tubular stent according to claim 8, wherein the strut comprises a bent portion disposed between the first and second portions, wherein the bent portion is curved.

13. The tubular stent according to claim 8, wherein the first portion comprises a first radial side which defines a curved section substantially adjacent to a first primary column in the circumferentially adjacent pair of primary columns, and the second portion comprises a first radial side which defines a curved section substantially adjacent to the second primary column in the adjacent pair of primary columns.

14. The tubular stent according to claim 1, wherein the struts have a width between 10 and 1,000 μm.

15. The tubular stent according to claim 1, wherein the primary columns comprise a proximal end and a distal end, wherein the proximal ends of the primary columns define the proximal end of the stent and the distal ends of the primary columns are spaced apart from the distal end of the stent.

16. The tubular stent according to claim 1, wherein the primary columns have a width between 10 and 1,000 μm.

17. The tubular stent according to claim 1, wherein a first non-linear strut is disposed between each pair of circumferentially adjacent primary columns, and is substantially adjacent to the proximal ends thereof, and a second non-linear strut is disposed between each pair of circumferentially adjacent primary columns, and is substantially adjacent to the distal ends thereof.

18. The tubular stent according to claim 1, wherein the stent comprises at least three secondary elongate columns disposed around the circumference of the stent, and substantially parallel to the longitudinal axis thereof, wherein the secondary columns intersect the struts.

19. The tubular stent according to claim 18, wherein the secondary columns comprise a proximal end and a distal end, wherein the distal ends of the secondary columns define the distal end of the stent, and the proximal ends of the secondary columns are spaced apart from the proximal end of the stent.

20. The tubular stent according to claim 1, wherein the stent consists of the primary columns and the struts, and optionally a drug-eluting coating and/or a polymer or fabric coating disposed around the outer circumference of the stent.

21. The tubular stent according to claim 1, wherein the stent comprises a polymeric material and/or a metal.
